(19) United States
(12) Patent Application Publication
(10) Pub. No.: US 2012/0115130 A1
(43) Pub. Date: May 10, 2012

(54) METHOD FOR DETECTING TARGET CELL

(75) Inventors: Toshihiko Imai, Edogawa-ku (JP); Masaaki Miyaji, Ibaraki (JP)

(73) Assignee: JSR CORPORATION, Tokyo (JP)

(21) Appl. No.: 13/378,133

(22) PCT Filed: Jun. 16, 2010
(86) PCT No.: PCT/JP10/60208

§ 371 Date: Jan. 24, 2012

(30) Foreign Application Priority Data
Jun. 16, 2009 (JP) 2009-142929
Nov. 13, 2009 (JP) 2009-259518

(51) Int. Cl. G01N 21/75; C12Q 1/68; G01N 27/72

(52) U.S. Cl. 435/6.1; 435/7.2

(57) ABSTRACT

A target cell detection method includes performing a test measurement on a dispersion to obtain a measurement result 1, the test measurement being an optical or electromagnetic measurement, and the dispersion including labeled particles and target cells, the labeled particles being particles on each of which a substance that specifically binds to a specific molecule present on a surface of each of the target cells is immobilized, performing measurement that is identical with the test measurement on a dispersion that includes the target cells, but does not include the labeled particles to obtain a measurement result 2, and comparing the measurement result 1 with the measurement result 2.

### TECHNICAL FIELD

The present invention relates to a target cell detection method.

### BACKGROUND ART

A method that performs a test on cells contained in a body fluid (i.e., cell dispersion) (e.g., blood, bone marrow, or lymph), and screens specific cells (e.g., cells that relate to a specific disease) that may be present in the cell dispersion, has been widely used. For example, flow cytometry or the like utilizes the fact that test target cells include a specific molecule (marker) (hereinafter may be referred to as a cell surface marker, a cell surface antigen, or a CD antigen) on the surface thereof, and detects cells that include such a cell surface marker (see JP-A-2008-187932 and JP-T-2008-538609). When the cell dispersion is a body fluid, the analysis results are used as data for diagnosis of a disease, for example. Note that a test target cell that is included in a cell dispersion and includes a specific cell surface marker is hereinafter referred to as “target cell”.

A flow cytometer is used for flow cytometry. Specifically, the cell surface marker of the test target cells is labeled with a labeled molecule such as a fluorescence-labeled monoclonal antibody (fluorescent antibody technique) or an enzyme-labeled monoclonal antibody (immunoenzymatic technique), or fluorescent particles or fluorescent inorganic semiconductor nanoparticles to which a labeled molecule binds (JP-A-2004-077389), and the flow cytometer discriminates the target cells by utilizing such a label. The labeled monoclonal antibody (MoAb) may be used to discriminate cells (e.g., lymph, monocyte, bone marrow, or megakaryocyte), and makes it possible to analyze the differentiation and the functions of the cells. The cell dispersion can be objectively and determinately characterized using the flow cytometer. Therefore, the analysis results may be used as reliable data for a doctor or the like to diagnose a disease.

However, the flow cytometer used for flow cytometry is expensive as compared with a blood cell counter, and the labeled monoclonal antibody used as a reagent is also expensive. Therefore, the flow cytometer is generally provided in special testing institutions, and has not been widely provided in hospitals and clinics. At present, a special institution (e.g., commercial laboratory) normally implements flow cytometry on commission, and it takes time to obtain the results. This makes it difficult to promptly provide data for a doctor or the like to diagnose a disease.

The method disclosed in JP-A-2008-187932 that immobilizes an antibody on a substrate, a plate, or the like, and causes cells to be adsorbed thereon can merely separate the target cell or determine the presence or absence of the target cells. It is necessary to reliably cause an antibody immobilized on a substrate or the like to come in contact with the target cells in order to determine the amount of the target cells and the ratio of the target cells to other cells using the method disclosed in JP-A-2008-187932. Moreover, it may be difficult to count the number of the target cells.

A hematology analyzer that is widely used for clinical laboratory may classify leukocytes into a granulocyte, a lymphocyte, and a monocyte (hereinafter referred to as “3-part WBC differential hematology analyzer”), or classify leukocytes into an eosinophil, a neutrophil, a basophil, a lymphocyte, and a monocyte (hereinafter referred to as “5-part WBC differential hematology analyzer”), and measure the ratio of the number of the respective cells. Such a hematology analyzer includes a mechanism that applies laser light to a sample, and detects the intensity of scattered light (e.g., forward scattered light or side scattered light), and can obtain information that reflects the size and the internal structure of the cells together with the blood cell count. For example, the ratio of the respective leukocytes can be determined using such a hematology analyzer. This makes it possible to detect abnormalities (e.g., an abnormal ratio of the respective leukocytes, or an increase in immature leukocytes) in a patient who suffers from leukemia. However, the results may differ depending on each hematology analyzer, and it is considered that a blood sample cannot necessarily be sufficiently characterized based only on the measurement results obtained using such a hematology analyzer.

Therefore, a method that detects the target cells by easily and accurately acquiring information about test target cells that are included in a cell dispersion and include a specific cell surface marker has been desired.

### SUMMARY OF INVENTION

### Technical Problem

An object of the invention is to provide a simple and accurate target cell detection method. Such a method may make it possible to detect the target cells without using flow cytometry, and easily analyze the size and the like of the target cells, and may be useful for testing a cell dispersion (e.g., blood) during clinical examination or the like.

### Solution to Problem

The invention was conceived in order to achieve the above object, and may be implemented as described below (see the following aspect (embodiment) or application example).

### Application Example 1

According to one aspect of the invention, there is provided a method for detecting target cells, the method including:

performing a test measurement on a dispersion to obtain a measurement result 1, the test measurement being an optical or electromagnetic measurement, and the dispersion including labeled particles and the target cells, the labeled particles being particles on each of which a substance that specifically binds to a specific molecule present on a surface of each of the target cells is immobilized;

performing measurement that is identical with the test measurement on a dispersion that includes the target cells, but does not include the labeled particles to obtain a measurement result 2; and

comparing the measurement result 1 with the measurement result 2.

### Application Example 2

In the target cell detection method according to Application Example 1, the test measurement may not include fluorescence measurement.

### Application Example 3

In the target cell detection method according to Application Example 1 or 2, the test measurement may include measurement of scattered light.

### Application Example 4

In the target cell detection method according to any one of Application Examples 1 to 3, the test measurement may include measurement of the number and the size of the cells by an electromagnetic method.

### Application Example 5

In the target cell detection method according to any one of Application Examples 1 to 4, each of the labeled particles may include a polar group.

### Application Example 6

In the target cell detection method according to Application Example 5, the polar group may be at least one group selected from a hydroxyl group, an epoxy group, a carboxyl group, an alkylene oxide group, a keto group, and a substituted or unsubstituted amino group.

### Application Example 7

In the target cell detection method according to any one of Application Examples 1 to 6, the test measurement may include measuring a binding amount of each of the target cells and the labeled particles, and determining a distribution of the number of the target cells with respect to the binding amount.

### Application Example 8

In the target cell detection method according to any one of Application Examples 1 to 7, the test measurement may be performed on a plurality of measurement items.

### Application Example 9

In the target cell detection method according to any one of Application Examples 1 to 8, the dispersion may include a body fluid.

### Application Example 10

In the target cell detection method according to any one of Application Examples 1 to 9, the test target cells may be blood cells including leukocytes, and the test measurement may be performed by using a hematology analyzer that classifies leukocytes into a granulocyte, a lymphocyte, and a monocyte, or a hematology analyzer that classifies leukocytes into an eosinophil, a neutrophil, a basophil, a lymphocyte, and a monocyte.

### Application Example 11

In the target cell detection method according to any one of Application Examples 1 to 10, the labeled particles may include labeled particles 1 and labeled particles 2, a substance that specifically binds to a first antigen being immobilized on each of the labeled particles 1, and a substance that specifically binds to a second antigen being immobilized on each of the labeled particles 2.

### Advantageous Effects of Invention

The target cell detection method makes it possible to easily and promptly detect the target cells, and analyze the size and the like of the target cells. This makes it possible to easily and promptly provide data for diagnosing a disease when the cell dispersion is blood or the like. Since the target cell detection method makes it possible to perform measurement that has been performed using flow cytometry using a 5-part WBC differential hematology analyzer, a 3-part WBC differential hematology analyzer, or the like, the target cells included in the cell dispersion can be more easily analyzed.

### BRIEF DESCRIPTION OF DRAWINGS

FIG. 1 is a scattergram illustrating an example of a blood analysis step.

FIG. 2 is a scattergram illustrating an example of an analysis step of a cell analysis method according to the examples.

### DESCRIPTION OF EMBODIMENTS

Several preferred embodiments of the invention are described below. Note that the invention is not limited to the following embodiments. Various modifications may be made of the following embodiments without departing from the scope of the invention.

### 1. Target Cell Detection Method

A target cell detection method according to one embodiment of the invention includes a step 1 that includes performing a test measurement on a dispersion to obtain a measurement result 1, the test measurement being optical or electromagnetic measurement, and the dispersion including labeled particles and test target cells, the labeled particles being particles on which a substance that specifically binds to a specific molecule present on a surface of cells is immobilized, a step 2 that includes performing a test measurement that is identical with the test measurement performed in the step 1 on a dispersion that includes the test target cells, but does not include the labeled particles to obtain a measurement result 2, and a step 3 that includes comparing the measurement result 1 with the measurement result 2.

### 1.1. Cell Dispersion and Cell Dispersion Preparation Method

The cell dispersion prepared in one embodiment of the invention is not particularly limited as long as at least the target cells are dispersed (suspended) in a liquid. Examples of the cell dispersion include a body fluid of an animal (e.g., human) (e.g., blood, lymph, tissue fluid, and celomic fluid), and the like. A cell dispersion prepared by diluting a body fluid with an appropriate dispersion medium (e.g., isotonic buffer solution) may also be used. The cell dispersion is not limited to a cell dispersion derived from a living body, but may be a cell dispersion prepared by dispersing cells in a liquid for the purpose of tests, study, or the like. The dispersion medium for the cells is not particularly limited. The dispersion medium is normally water, plasma, or the like. The dispersion medium may be an organic solvent such as glycerol or an alcohol. The cell dispersion may include salt, a buffer, or a drug as a solute.

Specific examples of the target cells according to one embodiment of the invention include leukocytes, red blood cells, platelets, artificial cells (e.g., genetically-modified cells), cells that include an antigen on the surface thereof (e.g., cancer cells), and the like. The cell surface marker included in the target cells may be at least one of proteins, sugar chains, glycoconjugates, and lipids, for example. A single target cell may include different types of cell surface markers. The type of the cell surface marker is not particularly limited. Examples of the proteins include receptors, CD antigens (in accordance with the cluster of differentiation (CD)), and the like. The sugar chain may be at least one of glycoprotein sugar chains, glycolipid sugar chains, glycosaminoglycan sugar chains, and polysaccharide-derived oligosaccharide chains, for example. Examples of the glycoconjugates include biological polymers that include a sugar chain. The glycoconjugate may be at least one of glycoproteins (including glycopeptides), proteoglycans, and glyco lipids, for example.

The cell dispersion may include the target cells and cells other than the target cells. For example, when the cell dispersion is blood, and the target cells are leukocytes, red blood cells and platelets correspond to the cells other than the target cells. When the cell dispersion is blood, and the target cells are red blood cells, leukocytes and platelets correspond to the cells other than the target cells. When cells that include a specific cell surface marker due to infection with a virus that causes a specific disease are the target cells, a sample of normal cells that does not include the cells infected with the virus may not include the target cells.

The concentration of cells dispersed in the cell dispersion is not particularly limited. For example, when the cell dispersion is blood, and the target cells are leukocytes, the concentration of cells dispersed in the cell dispersion is preferably 1000 to 20,000 cells/microliter. Blood or an arbitrary body fluid may optionally be diluted, and may be used as the cell dispersion. When preparing a cell dispersion, the concentration of the target cells dispersed in the cell dispersion may be about 10 to 1×10⁷ cells/microliter.

### 1.2. Labeled Particles and Labeled Particle Preparation Method

The labeled particles used in one embodiment of the invention is prepared by immobilizing a substance that specifically binds to a specific molecule present on the surface of cells on base particles described below.

### 1.2.1. Base Particles

The shape of the base particles is not particularly limited. For example, the base particles may have a spherical shape, a spheroidal shape, a columnar shape, an amorphous shape, or the like. The entire base particles may not have a uniform shape. The base particles may be a mixture of particles that differ in shape. Spherical polymer particles can be easily produced by emulsion polymerization or the like. When the particles have a uniform shape, scattered light obtained by applying laser light has a characteristic feature, and the particles can be easily specified by optical observation, for example.

The number average particle size of the base particles is preferably 0.04 to 10 micrometers, more preferably 0.5 to 10 micrometers, and particularly preferably 1 to 5 micrometers. If the number average particle size (diameter) of the base particles is less than 0.04 micrometers, the measurement result may not be clearly changed when the particles are adsorbed on the target cells. When the cell dispersion is blood, and the target cells are leukocytes, the particles have a size equal to or larger than that of the target cells if the number average particle size of the base particles exceeds 10 micrometers. In this case, it may be difficult to distinguish the particles that are not adsorbed on the target cells from the target cells. Moreover, the measurement result may be changed to a large extent when the particles are adsorbed on the target cells, so that it may be difficult to appropriately acquire information in the measurement step. If the number average particle size of the base particles is 0.5 micrometers or more, the test measurement can be easily performed by a light scattering method.

The number average particle size is determined by a light scattering method or a light blocking method as the number average particle size based on polystyrene particles, or determined by electron microscopy. If the number average particle size of the base particles determined by any of these methods is within the above range, the base particles may be suitably used. It is preferable to use the light scattering method when the particle size is about 0.04 to 1 micrometer, it is preferable to use the light blocking method when the particle size is about 1 to 5 micrometers, and it is preferable to use electron microscopy when the particle size is about 5 micrometers or more.

The material for the base particles is not particularly limited. The base particles may be organic particles or inorganic particles. Examples of the organic particles include particles formed of polystyrene, polylactic acid, acryl, polyethylenimine, agarose, an iminodiacetic acid chelate, magnetic latex, magnetic polylactic acid, magnetic dextran, magnetic chitosan, magnetic agarose, magnetic polyethylenimine, and the like. Examples of the inorganic particles include silica, magnetic silica, iron oxide, inorganic semiconductor particles, and the like. The base particles may a mixture of particles that differ in material. Specifically, two or more types of organic particles or inorganic particles, or organic particles and inorganic particles may be used in combination as the base particles.

It is preferable that the base particles include a polar group. It is more preferable that the base particles include a polar group on the surface thereof. The polar group is preferably at least one group selected from a hydroxyl group, an epoxy group, a carboxyl group, an alkylene oxide group, a keto group, and a substituted or unsubstituted amino group. The particles become hydrophilic as a result of introducing a polar group into the surface of the particles, so that non-specific adsorption of the cells in the cell dispersion can be suppressed. This makes it possible to improve the reliability of the measurement results.

The base particles that include a hydrophilic group may be prepared by subjecting a monomer that includes a hydrophilic group (hydrophilic monomer) to emulsion polymerization together with another raw material, or coating the particles with a monomer unit that includes a hydrophilic monomer, and polymerizing the monomer unit, for example.

Examples of the hydrophilic monomer include (meth)acrylates that include a hydrophilic functional group, such as glycerol acrylate, glycerol methacrylate, 2-hydroxyethyl acrylate, 2-hydroxyethyl methacrylate, methoxyethyl acrylate, methoxyethyl methacrylate, polyethylene glycol monoacrylate, polyethylene glycol monomethacrylate, acrylic acid, methacrylic acid, acrylamide, methacrylamide, N-methylolacrylamide, N-methylolmethacrylamide, diacetoneacrylamide, allyl glycidyl ether, glycidyl acrylate, glycidyl methacrylate, and the like. When polymerizing a monomer that includes an epoxy group (e.g., glycidyl acrylate or glycidyl methacrylate), a hydroxyl group (i.e., polar group) is produced via hydrolysis. Therefore, such a monomer may also be suitably used.

The base particles may be colored to display a color (e.g., red, blue, or green) in the visible region. This makes it possible to improve the visibility of the particles when observing the particles using an optical microscope, for example. As a result, the target cells can be easily searched using a microscope when the number of target cells (i.e., cells to which the particles bind) in the cell dispersion is small, for example.

1.2.2. Substance that Specifically Binds to Specific Molecule Present on Surface of Cells

Examples of the substance that specifically binds to a specific molecule present on the surface of cells include, but are to limited to, protein molecules such as an antibody (e.g., monoclonal antibody and polyclonal antibody), an aggregate of protein molecules, a Fab′ fragment of an antibody, polynucleotides, and the like. An antibody that belongs to any of the subclasses IgG IgM, IgA, IgEs, IgD, and the like may be used.

When the cell surface marker is a CD antigen, it is preferable that a monoclonal antibody (anti-CD antibody) that specifically binds to the CD antigen be immobilized on the base particles. Specific examples of the anti-CD antibody include an anti-CD3 antibody (hereinafter may be abbreviated as “CD3 antibody”), an anti-CD4 antibody (hereinafter may be abbreviated as “CD4 antibody”), an anti-CD8 antibody (hereinafter may be abbreviated as “CD8 antibody”), and the like. These anti-human CD monoclonal antibodies may be prepared using a mouse, rat, rabbit, sheep, or the like.

### 1.2.3. Labeled Particle Preparation Method

The substance that specifically binds to a specific molecule present on the surface of cells may be immobilized on the base particles by an arbitrary method. For example, the substance may be physically immobilized on the base particles via adsorption or the like, or may be chemically immobilized on the base particles via a covalent bond, a hydrogen bond, or the like. The substance (e.g., antibody) may be immobilized directly on the base particles (hereinafter may be referred to as “direct method”), or may be immobilized on the base particles in a state in which another substance is interposed between the base particle and the substance (e.g., antibody) (hereinafter may be referred to as “indirect method”).

For example, an antibody may be immobilized directly on the base particles by causing the Fc region of the antibody to be covalently bonded to the functional group on the surface of the particles using a coupling agent. A carbodiimide such as 1-ethyl-3-(3-dimethylaminopropyl)-carbodiimide hydrochloride (EDC) or the like may be used as the coupling agent. When using EDC as the coupling agent, the antibody can be immobilized directly on the base particles via a covalent bond formed by a carbodiimide coupling reaction. A blocking agent may be used in combination with the coupling agent. Bovine serum albumin (BSA), gelatin, skimmed milk, egg albumin, or the like may be used as the blocking agent.

The amount of the antibody that is immobilized directly on the base particles is preferably 0.1 to 100 micrograms/mg based on the weight of the base particles.

An antibody may be immobilized indirectly on the base particles by causing a primary antibody to bind to the base particles via a secondary antibody. In this case, the primary antibody specifically binds to the cell surface marker, and the secondary antibody specifically binds to the primary antibody. An antibody may also be immobilized indirectly on the base particles by causing a protein (e.g., protein G or protein A) that specifically binds to the Fc region of IgG, or a derivative thereof, to bind to the base particles, and causing an antibody that specifically binds to the cell surface marker to bind to the protein or a derivative thereof, for example.

When using the primary antibody and the secondary antibody, an antibody corresponding to the animal species from which the immunoglobulin of the primary antibody is derived is preferably selected as the secondary antibody. For example, when the primary antibody is an anti-human CD antigen monoclonal antibody that is derived from a mouse (hereinafter may be referred to as “anti-human CD antigen mouse antibody”), it is preferable to select an anti-mouse immunoglobulin antibody (e.g., anti-mouse IgG antibody) as the secondary antibody. For example, when the primary antibody is an anti-human CD antigen mouse IgG antibody, it is preferable to select an anti-mouse IgG antibody as the secondary antibody. In this case, the anti-mouse IgG antibody is preferably derived from a rat, rabbit, sheep, or the like.

The amount of the anti-mouse IgG antibody that is bound to the base particles as the secondary antibody is preferably 0.1 to 10 micrograms/mg based on the weight of the base particles. The amount of protein A or protein G that is bound to the base particles is preferably 0.1 to 100 micrograms/mg based on the weight of the base particles. In such a case, the amount of the primary antibody is preferably 0.1 to 100 micrograms/mg based on the weight of the particles.

The primary antibody may be bound to the base particles at a high rate via protein G or protein A, and an anti-mouse IgG antibody or the like may be bound to the protein G or protein A as a masking agent in order to suppress non-specific adsorption. In such a case, the amount of the anti-mouse IgG antibody or the like is determined depending on the particle size of the particles and the binding amount of protein A or protein G, but is preferably 0.1 to 100 micrograms/mg based on the weight of the particles.

The labeled particles are used in a state in which the labeled particles are dispersed in a dispersion medium such as a physiological saline solution or a buffer (e.g., boric acid buffer, EDTA buffer, Tris buffer, or phosphate buffer).

### 1.3. Step 1

The step 1 includes performing the test measurement on a dispersion that includes the labeled particles and the test target cells to obtain a measurement result.

The measurement result obtained by the step 1 is referred to as “measurement result 1”. When the target cells are present in the cell dispersion, the labeled particles specifically bind to the target cells via an antigen-antibody reaction or the like. The labeled particles do not specifically bind to cells other than the target cells. Therefore, only the target cells bind to one or more labeled particles in the step 1.

### 1.3.1. Dispersion of Cells and the Like

The concentration of the labeled particles included in the dispersion used in the step 1 is adjusted depending on the type of the cell dispersion, but is preferably 1×10² to 9×10¹⁰ per microliter. When the cell dispersion is blood, and the target cells are leukocytes that include a specific cell surface marker, the probability that the labeled particles bind to the target cells decreases to a large extent even if the incubation time is sufficient if the concentration of the labeled particles is less than 1×10² per microliter (i.e., one-tenth of the leukocyte count in normal blood). When the cell dispersion is blood, and the target cells are leukocytes that include a specific cell surface marker, a large number of particles that do not bind to the target cells are present in the cell dispersion if the concentration of the labeled particles exceeds 9×10¹⁰ per microliter (i.e., the number of particles is significantly larger than the leukocyte count). As a result, the test measurement may be hindered, so that it may be difficult to accurately measure the target cells. When the cell dispersion is blood, and is supplied as a dispersion in which the labeled particles are dispersed at a concentration of about 1 g/l, it is preferable to add the dispersion of the particles in an amount of about 10 microliters per 100 microliters of blood.

When using colored labeled particles, different types of antibodies or the like may respectively be immobilized on the labeled particles that differ in color. This makes it possible to modify the target cells with the labeled particles that differ in color, so that the antigen included in the target cells can be easily identified by observation using an optical microscope or spectral measurement, for example. This makes it possible to more easily discriminate the target cells.

When the cell dispersion includes the target cells that include a plurality of types of cell surface markers on the surface thereof, first labeled particles and second labeled particles may be added to the cell dispersion, a first substance that specifically binds to a first cell surface marker among the plurality of types of cell surface markers being immobilized on the first labeled particles, and a second substance that specifically binds to a second cell surface marker among the plurality of types of cell surface markers being immobilized on the second labeled particles, for example. This makes it possible to perform the test measurement corresponding to each cell surface marker.

### 1.3.2. Test Measurement

The test measurement is performed on the cell dispersion by an optical or electromagnetic method. The term “electromagnetic method” used herein refers to a human-imperceptible method such as an electronic method and a magnetic method. For example, the test measurement may be implemented by measuring the number and the size of cells by an optical method (e.g., light scattering method, light blocking method, or fluorometry), an electromagnetic method based on the Coulter principle (Beckman Coulter, Inc.), or the like. It is preferable to use the optical method (excluding fluorometry), the electromagnetic method, or a combination of the optical method (excluding fluorometry) and the electromagnetic method. The light scattering method is preferable as the optical method (excluding fluorometry). When using fluorometry, it is necessary to use labeled particles that can emit fluorescence, and a method such as flow cytometry. Therefore, the equipment/reagent cost increases, for example.

The test measurement may be implemented by one test measurement method, or may be performed on a plurality of test measurement items. When performing the test measurement on a plurality of test measurement items, the resulting measurement results may be utilized in combination. For example, when measuring the size and the number of cells by the light scattering method or the like, the correlation between the size and the number of cells can be determined (i.e., the amount of information obtained increases). The binding amount of respective target cells and the labeled particles may be measured, and the distribution of the number of the target cells with respect to the binding amount may be determined.

Examples of the light scattering method include a forward scattered light intensity measurement method and a side scattered light intensity measurement method.

### (1) Forward Scattered Light Intensity Measurement Method

The term “forward scattered light” used herein refers to laser light that has been scattered forward with respect to the laser light axis due to cells. The intensity of forward scattered light is proportional to the projection area of the cells. Specifically, the intensity of forward scattered light increases when applying laser light to large cells, and decreases when applying laser light to small cells. Therefore, the size of the cells and the like can be estimated by measuring the intensity of forward scattered light.

When the labeled particles have bound to the target cells, the intensity of forward scattered light may increase as compared with the intensity of forward scattered light obtained when the target cells are not modified. Therefore, it is observed that the modified target cells have an increased apparent size when measuring the intensity of forward scattered light. Specifically, the apparent size of the target cells can be estimated by measuring the intensity of forward scattered light. This makes it possible to evaluate whether or not the labeled particles bind to the target cells, the number of labeled particles that bind to the target cells, and the like.

It is also possible to measure the intensity of forward scattered light that has been scattered over a wider angle with respect to the laser light axis. The intensity of wide-angle forward scattered light includes information such as the presence or absence, the number, and the density of granules in the cells. There is a tendency that the internal structure of the cells is complex when the intensity of wide-angle scattered light is high, and is simple when the intensity of wide-angle scattered light is low. Therefore, information about the presence or absence, the number, and the density of granules in the cells and the structure the cells can be estimated by measuring the intensity of wide-angle scattered light.

When analyzing leukocytes by measuring the intensity of forward scattered light, it is preferable that the labeled particles have a number average particle size of 2 to 5 micrometers.

### (2) Side Scattered Light Intensity Measurement Method

The term “side scattered light” used herein refers to laser light that has been scattered in the direction perpendicular to the laser beam axis due to cells. The intensity of side scattered light changes due to a scatterer that is smaller than the cells. Therefore, the intensity of side scattered light includes information such as the degree of lobulation of the nucleus in the cells in the same manner as the intensity of wide-angle forward scattered light. Specifically, there is a tendency that the internal structure of the cells is complex when the intensity of side scattered light is high, and is simple when the intensity of side scattered light is low. Therefore, information about the presence or absence and the number of cells having a small degree of lobulation can be normally estimated by measuring the intensity of side scattered light.

When the labeled particles have bound to the target cells, the intensity of side scattered light may increase as compared with the intensity of side scattered light obtained when the target cells are not modified. Therefore, a relative change in the internal structure of the modified target cell with respect to the unmodified target cells is observed when measuring the intensity of side scattered light. Specifically, the apparent degree of lobulation of the nucleus in the target cells can be estimated by measuring the intensity of side scattered light. This makes it possible to evaluate whether or not the labeled particles bind to the target cells, the number of labeled particles that bind to the target cells, the complexity of the internal structure of the cells, and the like.

When analyzing leukocytes by measuring the intensity of side scattered light, it is preferable that the labeled particles have a number average particle size of 300 nm to 5 micrometers.

### (3) Side Fluorescence Intensity Measurement Method

The term “side fluorescence” used herein refers to fluorescence that is emitted in the direction perpendicular to the laser light axis when laser light is incident on a fluorescent substance of cells. The intensity of side fluorescence changes depending on the amount of fluorescent substance that is included in (or binds to) the cells. The intensity of side fluorescence may be measured as described below, for example.

When staining a cell component with a fluorescent substance that specifically binds to DNA or RNA, the stained cells absorb laser light, so that the fluorescent substance emits fluorescence. In this case, the intensity of side fluorescence changes depending on the amount of DNA or RNA stained with the fluorescent substance. Therefore, the intensity of side fluorescence includes information about the amount of DNA or RNA included in the cells.

When causing the labeled particles including a fluorescent substance to bind to the surface of the target cells, the fluorescent substance emits fluorescence when applying laser light to the modified target cells. In this case, the intensity of side fluorescence includes information about the amount (number) of labeled particles that bind to the target cells.

Propidium iodide, ethidium bromide, acridine orange, or the like may be used as the fluorescent substance. A plurality of fluorescent substances may be used in combination. The fluorescent substance may be bound to the surface of the particles, or may be added together with a monomer when synthesizing the particles so that the fluorescent substance is incorporated in the particles, for example.

The intensity of side fluorescence may optionally be measured using a 5-part WBC differential hematology analyzer or the like.

The test measurement is preferably performed when 30 seconds to 60 minutes has elapsed after preparing a dispersion by mixing the cells and the labeled particles. When the cell dispersion is blood, coagulation of blood or the like may occur if the test measurement is performed when a longer time has elapsed. A flow cytometer utilizes an optical method (e.g., fluorometry). A blood cell counter that utilizes the Coulter principle counts the number of cells that have passed through pores in an electric field by utilizing a phenomenon in which an electrolytic solution is substituted with the cells when the cells pass through the pores, so that a change in electrical resistance, impedance, electromagnetic field, and the like occurs. In principle, the blood cell counter that utilizes the Coulter principle can measure each of a number of cells dispersed in the cell dispersion. Since a change in electrical resistance increases as the size of the cells that pass through the pores increases, the number, the size, and the volume of the cells in the cell dispersion and the like can be promptly measured using the blood cell counter. The diameter of cells for which the number and the like can be measured using the blood cell counter is normally about 1 to 30 micrometers.

A 3-part WBC differential hematology analyzer and a 5-part WBC differential hematology analyzer include a blood cell counter, a laser irradiation device, a scattered light-receiving device, and the like. The 3-part WBC differential hematology analyzer and the 5-part WBC differential hematology analyzer can measure the intensity of scattered laser light corresponding to each of a number of cells dispersed in the cell dispersion. Examples of the intensity of scattered light to be detected include the intensity of forward scattered light, the intensity of side scattered light, the intensity of side fluorescence, and the like.

In the step 1, the measurement result may be obtained as a numerical value (e.g., the intensity of scattered light). When using a plurality of test measurement methods in combination, the correlation between the measured values obtained by the respective test measurement methods may be indicated using a scattergram, for example. For example, a numerical value that indicates the number of cells in the cell dispersion and a numerical value that indicates the size of cells in the cell dispersion may be indicated using a scattergram, or a numerical value that indicates the size of cells in the cell dispersion and a numerical value that indicates information about the internal structure of cells (e.g., the amount (number) of granules) may be indicated using a scattergram. The scattergram may be a three or higher dimensional scattergram. Since such a scattergram can advantageously indicate the features (characteristics) of the cell dispersion, such a scattergram is useful as data for diagnosis by a doctor or the like.

Information about the presence or absence of the target cell in the cell dispersion, the number of the target cells, and the like can be obtained from the measurement result obtained by the step 1. This makes it possible to classify the target cells based on the degree of modification, and characterize the cell dispersion based on the classification, for example.

The analysis process performed in the analysis step may include comparing the measurement result for the target cells modified by the modification step with the measurement result for the unmodified target cells. This makes it possible to determine the amount of antigen present on the surface of the target cells, and characterize the cell dispersion based on the analysis result, for example.

The analysis process performed in the analysis step may include comparing the information obtained (measured) before the modification step with the information obtained (measured) after the modification step. This makes it possible to specify and classify the target cells at the same time, and characterize (discriminate) the cell dispersion based on the analysis result, for example.

The cells can be classified based on the type of antigen present on the surface of the cells included in the cell dispersion, and the cell dispersion can be characterized based on the analysis result by performing the above analysis step.

### 1.4. Step 2

The step 2 includes performing the same test measurement as that performed in the step 1 on a dispersion that includes the test target cells, but does not include the labeled particles to obtain a measurement result. The measurement result obtained by the step 2 is referred to as “measurement result 2”. The expression “test measurement identical with the test measurement performed in the step 1” refers to a test measurement that is identical with the test measurement performed in the step 1 as to at least one of the measurement item and the measurement conditions.

The step 2 differs from the step 1 in that the dispersion does not include the labeled particles. The cell dispersion used in the step 2 is identical with the cell dispersion used in the step 1 other than the above point. Note that the step 2 may be performed before the step 1, or may be performed after the step 1.

### 1.5. Step 3

The step 3 includes comparing the measurement result 1 with the measurement result 2. The measurement result 1 and the measurement result 2 may be compared by an arbitrary method. For example, the measurement result 1 and the measurement result 2 may be compared as numerical value data, or may be compared using a scattergram or the like that indicates the resulting numerical value data. The measurement result 1 and the measurement result 2 may be compared quantitatively, or may be compared qualitatively (e.g., compared visually using a scattergram). The test measurement may be performed on a plurality of measurement items, and the correlation of each measurement item may be analyzed to compare the measurement result 1 with the measurement result 2. The step 3 may be performed using a blood cell counter, a 3-part WBC differential hematology analyzer, a 5-part WBC differential hematology analyzer, or a flow cytometer in the same manner as the steps 1 and 2.

### 1.6. Additional Step

The target cell detection method according to the invention may include an additional step other than the steps 1 to 3. Examples of the additional step include an observation step that includes observing the cell dispersion using an optical microscope. Specifically, a smear of the cell dispersion is prepared, and observed using an optical microscope. The smear may be prepared by a known method. When the cell dispersion is human blood, the incubation time elapsed until a blood smear is prepared after adding the particles on which an antibody is immobilized is preferably 30 seconds to 60 minutes. Note that the incubation time is not limited thereto.

The observation step may be provided to confirm information obtained by the measurement step, for example. In this case, whether or not the modified target cell is the target cell may be determined in the observation step, for example.

The observation step may be provided to obtain information other than the measurement result obtained by the measurement step. For example, when using colored particles corresponding to the antigen present on the surface of the target cells, information about the type of the cells can be more directly obtained by the observation step. In this case, the cells can be classified in more detail based on the antigen present on the surface of the cells. When observing the blood smear using a microscope, the objectivity of the morphological test can be improved by performing multicolor analysis using a plurality of markers (i.e., a plurality of colors), for example.

First labeled particles and second labeled particles that differ in color in the visible region may be used as the labeled particles, the target cells may be specified in the observation step using the first labeled particles and the second labeled particles as labels, and the target cells may be discriminated based on the number of first labeled particles and second labeled particles that bind to the target cells.

The target cells in the cell dispersion can be very easily classified by the target cell detection method according to the invention. Moreover, the target cells can be detected easily and promptly. This makes it possible to easily and promptly provide data for a doctor or the like to diagnose a disease when the cell dispersion is blood, for example.

### 2. Examples

The invention is further described below by way of examples. Note that the invention is not limited to the following examples.

### 2.1. Labeled Particle Preparation Example

### 2.1.1. Base Particles

A commercially-available product “MS300/Tosyl” (manufactured by JSR Corporation) was provided as base particles. The product “MS300/Tosyl” is formed of a magnetic latex and has a number average particle size determined by the light scattering method of 3 micrometers. The base particles were dispersed in a phosphate buffer at a concentration of 10 mg/ml. The base particles were used in each example.

### 2.1.2. Labeled Particle Preparation Example 1 (Direct Method)

The base particle dispersion was sufficiently mixed using a vortex mixer, and 1.0 ml (amount of particles: 10 mg) of the dispersion of the base particles was put in a microtube. The microtube was placed on a magnetic stand for about 1 minute, and the supernatant liquid was removed to concentrate the base particle dispersion. After the addition of 0.5 ml of a boric acid buffer (0.1M, pH: 9.5) (hereinafter referred to as “reaction buffer”), the particles were dispersed using a vortex mixer. The concentration operation and the dispersion operation using the reaction buffer were repeated twice. 0.5 ml of the reaction buffer was then added to the base particle dispersion, and the particles were dispersed using a vortex mixer to obtain a base particle dispersion A.

After the addition of 25 microliters of a 2 mg/ml anti-human CD4 antibody solution (manufactured by R&D Systems) to the base particle dispersion A, the mixture was stirred using a vortex mixer, and mixed upside down at 37° C. for 1 hour. After the addition of 2 microliters of a 10% bovine serum albumin (BSA) solution (manufactured by Aldrich) (blocking agent), the mixture was mixed upside down at 37° C. for 24 hours.

The microtube was placed on a magnetic stand for 1 minute, and the supernatant liquid was removed. After the addition of 0.5 ml of a physiological Tris buffer TBS-T (hereinafter referred to as “washing buffer”), the mixture was stirred using a vortex mixer. The supernatant liquid removal operation and the washing buffer addition operation were repeated three times.

After the addition of 1.0 ml of phosphate buffered saline (PBS) (hereinafter referred to as “storage buffer”), the mixture was stirred using a vortex mixer. The labeled particle dispersion thus obtained is hereinafter referred to as “PCD4”. The concentration of the labeled particles in the labeled particle dispersion PCD4 was 10 mg/ml.

### 2.1.3. Labeled Particle Preparation Example 2 (Indirect Method)

An anti-mouse IgG antibody was immobilized on the base particles in the same manner as in Labeled Particle Preparation Example 1, except that an anti-mouse IgG antibody (secondary antibody) was used instead of the anti-human CD4 antibody. The particle dispersion thus obtained is hereinafter referred to as “PIM”.

After the addition of 10 microliters of a 2 mg/ml anti-human CD4 antibody solution (manufactured by R&D Systems) to the particle dispersion PIM, the mixture was stirred using a vortex mixer, and mixed upside down at room temperature for 30 minutes. The microtube was placed on a magnetic stand for 1 minute, and the supernatant liquid was removed. After the addition of 0.5 ml of the washing buffer, the particles were dispersed using a vortex mixer. The supernatant liquid removal operation and the washing buffer addition operation were repeated three times. After the addition of 1.0 ml of the washing buffer, the particles were dispersed using a vortex mixer. The labeled particle dispersion thus obtained is hereinafter referred to as “PIMCD4”.

### 2.1.4. Labeled Particle Preparation Example 3 (Indirect Method)

Protein A was immobilized on the base particles in the same manner as in Labeled Particle Preparation Example 1, except that protein A was used instead of the anti-human CD4 antibody. The particle dispersion thus obtained is hereinafter referred to as “PPA”.

An anti-CD4 rabbit monoclonal antibody (manufactured by Cell Marque Corporation) was immobilized on the base particles in the same manner as in the primary antibody binding step in Labeled Particle Preparation Example 2. The labeled particle dispersion thus obtained is hereinafter referred to as “PPACD4”.
2.1.5. Labeled Particle Preparation Example 4 (indirect method)

A labeled particle dispersion “PPGCD4” was obtained in the same manner as in Labeled Particle Preparation Example 3, except that protein G was used instead of protein A.

### 2.2. Dispersion Preparation Example

2 ml of blood collected from the vein of a healthy individual using a blood collection tube to which EDTA was added was put in a microtube. 200 microliters of the labeled particle dispersion obtained in each of Labeled Particle Production Examples 1 to 4 was added to the microtube to obtain a dispersion used in each example. The mixture was then mixed upside down 20 times, allowed to stand at room temperature (15 to 25° C.) for 20 minutes, and used in each example. The concentration of the labeled particles in the dispersion was 5×10⁷ particles per microliter. As a control, 100 microliters of blood was used.

### 2.3. Measurement

The intensity of wide-angle forward scattered light and the electrical resistance based on the Coulter principle were measured using a blood analyzer “LH750”(manufactured by Beckman Coulter, Inc.) (5-part WBC differential hematology analyzer). The measured values were two-dimensionally plotted for each blood cell. The correlation between the distribution of the size of the blood cells and the distribution of the complexity of the internal structure of the blood cells was determined, and the blood cells were classified based on the plotted point (group). The lymphocyte ratio (LYMP %), the neutrophil ratio (NEUT %), the monocyte ratio (MONO %), the eosinophil ratio (EOS %), and the basophil ratio (BASO %) were calculated from the number of blood cells belonging to each group and the total number of blood cells. Note that the ratio of the respective blood cells refers to the ratio of the number of the respective blood cells to the total number of leukocytes contained in the blood. The results are given in Table 1. The change rate (%) in Table 1 was calculated by “ratio of respective blood cells in each example/ratio of respective blood cells in control×100(%)”

TABLE 1

| | | | Example 1 | | Example 2 | | Example 3 | | Example 4 | |
|---|---|---|---|---|---|---|---|---|---|---|
| | | | PCD4 | | PIMCD4 | | PPACD4 | | PPGCD4 | |
| | | Control | | Change | | Change | | Change | | Change |
| Labeled particle dispersion | | — | | rate (%) | | rate (%) | | rate (%) | | rate (%) |
| Granulocyte | Neutrophil ratio (%) | 64.3 | 68.3 | 6.2 | 69.8 | 8.6 | 67.7 | 5.3 | 68.4 | 6.4 |
| | Acidophil ratio (%) | 1.0 | 1.6 | 60.0 | 1.9 | 90.0 | 1.7 | 70.0 | 1.6 | 60.0 |
| | Basophil ratio (%) | 0.7 | 0.2 | −71.4 | 0.2 | −71.4 | 0.3 | −57.1 | 0.5 | −28.6 |

| | | | | | | | | | |
|---|---|---|---|---|---|---|---|---|---|
| Lymphocyte ratio (%) | 29.6 | 26.4 | −10.8 | 26.4 | −10.8 | 27.8 | −6.1 | 27.2 | −8.1 |
| Monocyte ratio (%) | 4.4 | 2.7 | −38.6 | 1.7 | −61.4 | 2.5 | −43.2 | 2.3 | −47.7 |

As is clear from Table 1, the lymphocyte ratio and the monocyte ratio decreased in each example as compared with the control, and the neutrophil ratio and the acidophil ratio (granulocyte ratio) increased in each example as compared with the control.

It is conjectured that the above results were obtained because the labeled particles on which the anti-human CD4 antibody was immobilized specifically bound to a CD4 antigen (formation of an immune complex) that is normally present on the surface of helper T cells (lymphocytes) and monocytes. Specifically, it is conjectured that the particles on which the anti-human CD4 antibody was immobilized specifically bound to lymphocytes, and the blood analyzer recognized the particles as granules in the cells. In other words, the blood analyzer recognized lymphocytes to which the particles (on which the anti-human CD4 antibody was immobilized) specifically bound as granulocytes (eosinophils or neutrophils) based on the size of the cells and the presence or absence of granules.

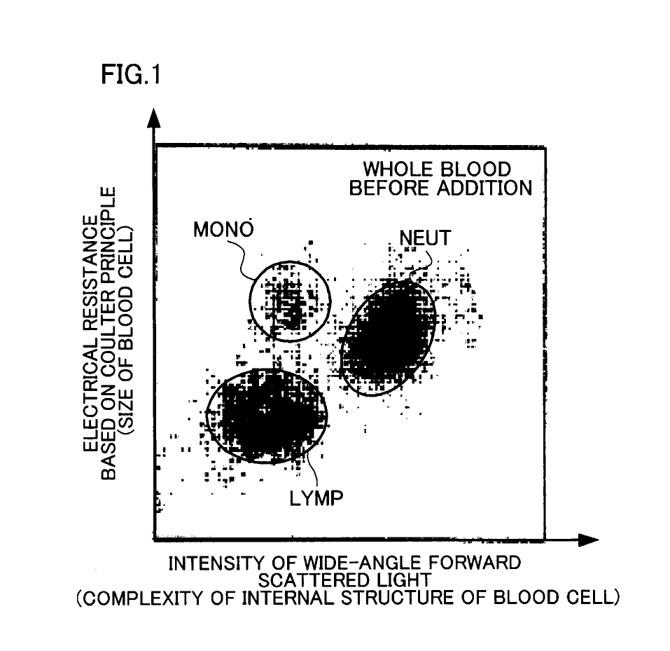

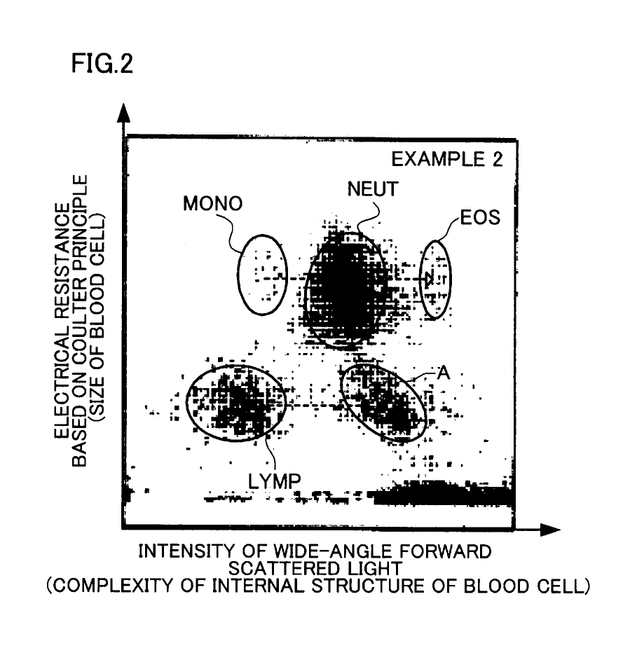

FIG. 1 is a scattergram obtained for the control, and FIG. 2 is a scattergram obtained in Example 2. The scattergrams illustrated in FIGS. 1 and 2 were obtained using a blood analyzer “LH750” (manufactured by Beckman Coulter, Inc.). As is clear from FIGS. 1 and 2, the blood analyzer determined that the lymphocytes (LYMP) on which the particles were adsorbed increased in the number of granule (horizontal axis) while maintaining the size (vertical axis), and moved the group classified as a lymphocyte to a position A under the group classified as a neutrophil (NEUT) within the scattergram. The group classified as a monocyte (MONO) also moved to the group classified as an eosinophil (EOS).

Since the particles on which an antibody that specifically binds to an antigen present on the surface of cells was immobilized were used in each example, the cell dispersion could be promptly characterized using a 5-part WBC differential hematology analyzer.

Note that the invention is not limited to the above embodiments. Various modifications and variations may be made without departing from the scope of the invention. For example, the invention includes various other configurations substantially the same as the configurations described in the embodiments (such as a configuration having the same function, method, and results, or a configuration having the same objective and results). The invention also includes a configuration in which an unsubstantial section (part) described in the embodiments is replaced by another section (part). The invention also includes a configuration having the same effects as those of the configurations described in connection with the above embodiments, or a configuration capable of achieving the same objective as that of the configurations described in connection with the above embodiments. The invention also includes a configuration in which a known technique is added to the configurations described in connection with the above embodiments.

### INDUSTRIAL APPLICABILITY

The invention makes it possible to very easily classify the target cells included in a cell dispersion, and promptly characterize the cell dispersion. Moreover, since a system other than a flow cytometer can be used, the cell dispersion can be easily and promptly characterized. The invention may also be applied to various hematology analyzer.

1. A method for detecting target cells, the method comprising:
(I) performing a first optical or electromagnetic measurement on a first dispersion to obtain a first measurement result, wherein the first dispersion comprises
the target cells, comprising a specific molecule present on a surface of the target cells, and
labeled particles, comprising a substance that specifically binds to the specific molecule;
(II) performing a second optical or electromagnetic measurement, which is identical to the first measurement, on a second dispersion to obtain a second measurement result, wherein the second dispersion comprises
the target cells, and
no labeled particles; and
(III) comparing the first measurement result to the second measurement result to detect target cells.

2. The method of claim 1, wherein the first measurement is not a fluorescence measurement.

3. The method of claim 1, wherein the first measurement comprises a measurement of scattered light.

4. The method of claim 1, wherein the first measurement comprises an electromagnetic measurement of a number and a size of the target cells.

5. The method of claim 1, wherein each of the labeled particles comprises a polar group.

6. The method of claim 5, wherein the polar group is at least one selected from the group consisting of a hydroxyl group, an epoxy group, a carboxyl group, an alkylene oxide group, a keto group, a substituted amino group, and an unsubstituted amino group.

7. The method of claim 1, wherein the first measurement comprises measuring a binding amount of each of the target cells and the labeled particles, and determining a distribution of the number of the target cells with respect to the binding amount.

8. The method of claim 1, wherein the first measurement is performed on a plurality of measurement items.

9. The method of claim 1, wherein the first and second dispersions comprise a body fluid.

10. The method of claim 1, wherein the target cells comprise blood cells comprising leukocytes, and wherein the first measurement employs
a hematology analyzer that classifies leukocytes into a cell type selected from the group consisting of a granulocyte, a lymphocyte, and a monocyte, or
a hematology analyzer that classifies leukocytes into a cell type selected from the group consisting of an acidophil, an eosinophil, a basophil, a lymphocyte, and a monocyte.

11. The method of claim 1, wherein the labeled particles comprise
a first group of labeled particles comprising a substance that specifically binds to a first antigen, and
(b) a second group of labeled particles comprising a substance that specifically binds to a second antigen.

12. The method of claim 1, wherein the target cells are at least one selected from the group consisting of leukocytes, red blood cells, platelets, genetically-modified cells, and cells comprising a surface antigen.

13. The method of claim 1, wherein the specific molecule present on a surface of each of the cells is at least one molecule selected from the group consisting of a protein, a sugar chain, a glycoconjugate, and a lipid.

14. The method of claim 13, wherein the protein is a receptor or a cluster-of-differentiation antigen.

15. The method of claim 13, wherein the sugar chain is at least one selected from the group consisting of a glycoprotein sugar chain, a glycolipid sugar chain, a glycosaminoglycan sugar chain, and a polysaccharide-derived oligosaccharide chain.

16. The method of claim 13, wherein the glycoconjugate is selected from the group consisting of a glycopeptide, a glycoprotein, a proteoglycan, and a glycolipid.

17. The method of claim 1, wherein the first dispersion comprises blood, the target cells are leukocytes, and a concentration of cells in the first dispersion is 1000 to 20,000 cells/microliter.

18. The method of claim 1, wherein a number average particle size of the labeled particles is 0.04 to 10 microns.

19. The method of claim 1, wherein the substance that specifically binds to the specific molecule is at least one substance selected from the group consisting of a protein molecule, an aggregate of protein molecules, a monoclonal antibody, a polyclonal antibody, a Fab′ fragment of an antibody, and a polynucleotide.
